(19) United States
(12) Patent Application Publication
(10) Pub. No.: US 2022/0296129 A1
(43) Pub. Date: Sep. 22, 2022

(54) ACTIVITY MONITORING SYSTEM

(71) Applicant: Repono Pty Ltd, Bardon (AU)

(72) Inventor: Nicholas Dodemont, Milton (AU)

(21) Appl. No.: 17/641,401

(22) PCT Filed: Jun. 30, 2020
(86) PCT No.: PCT/AU2020/050680

§ 371 Date: Mar. 8, 2022

(30) Foreign Application Priority Data
Sep. 9, 2019 (AU) 2019903328

(51) Int. Cl. A61B 5/11; A61B 5/00; A61B 5/0205; A61B 5/024; A61B 5/053; A61B 5/145

(52) U.S. Cl. CPC A61B 5/1118; A61B 5/6804; A61B 5/7246; A61B 5/0205; A61B 5/024; A61B 5/053; A61B 5/14517; A61B 5/14532; A61B 5/14546; A61B 5/4875; A61B 5/7275; A61B 2562/046; A61B 2576/00; A61B 2562/0261

(57) ABSTRACT

The present application relates to a system for monitoring a physical activity performed by a subject, the system including: a pose sensor, a garment and one or more processing devices. The pose sensor is configured to sense one or more pose parameters. The garment includes a number of arrays of electrodes positioned on the garment that contact skin of the subject and generate electrical signals indicative of electrical potentials within respective muscles of the subject, each array of electrodes including a plurality of electrodes arranged in a grid. The processing devices are configured to: determine one or more muscle activation patterns, at least partially determine the subject pose and, determine an activity indicator indicative of the physical activity at least partially based on the determined one or more muscle activation patterns and at least partially based on the determined subject pose.

### BACKGROUND OF THE INVENTION

The present invention relates to a system monitoring a physical activity performed by a subject, and in particular to a system for monitoring a physical activity performed by a subject using one or more pose sensors and at least one garment worn by the subject in use.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Whilst it is known to provide devices for monitoring activity, these are generally limited to detecting movement through the use of movement sensors, which does not accurately track muscle activity. Measuring muscle activity by recording electrical signals from muscles, in a process referred to as EMG (Electromyography) is known, this generally uses needle electrodes inserted into the muscles of the subject, which is therefore restrictive and therefore only used in limited situations.

WO2016/149751 describes a system for monitoring muscle activity of a biological subject, the system including at least one garment including a number of arrays of electrodes positioned on the garment so that when the garment is worn by a subject in use, the electrodes contact skin of the subject and generate electrical signals indicative of electrical potentials within respective muscles of the subject and at least one electronic processing device that processes signals from the electrodes in each electrode array to determine a muscle activation for parts of the respective muscles and uses the muscle activation to determine at least one muscle indicator indicative of muscle activity of the subject.

### SUMMARY OF THE PRESENT INVENTION

A system for monitoring a physical activity performed by a subject, the system including: a pose sensor configured to sense one or more pose parameters at least partially indicative of a subject pose; a garment including a number of arrays of electrodes positioned on the garment so that when the garment is worn by the subject, the electrodes contact skin of the subject and generate electrical signals indicative of electrical potentials within respective muscles of the subject, each array of electrodes including a plurality of electrodes arranged in a grid; and, one or more processing devices configured to: use signals from the electrodes to determine one or more muscle activation patterns; use the pose parameters to at least partially determine the subject pose; and, determine an activity indicator indicative of the physical activity at least partially based on the determined one or more muscle activation patterns and at least partially based on the determined subject pose.

In one example, the pose parameters are indicative of at least one of: a location of one or more limbs of the subject; a location of one or more joints of the subject; and, an angle of one or more joints of the subject.

In one example, the pose sensor includes an imaging device configured to capture one or more images of the subject when performing the physical activity and wherein the one or more processing devices are configured to: receive image data indicative of the one or more images from the pose sensor; and, analyse the image data to determine the pose parameters.

In one example, the one or more processing devices analyse the image data at least in part by identifying at least one of: an outline of the subject; a silhouette of the subject; segments of the subject; and, the subject.

In one example, the garment includes at least one marker and wherein the pose sensor is configured to detect the at least one marker.

In one example, the at least one marker includes at least one of: passive markers; active markers; and, a retroreflective marker.

In one example, the one or more processing devices are configured to determine at least one pose parameter using motion capture.

In one example, the one or more processing devices include: one or more central processing units; and, one or more tertiary processing units.

In one example, the one or more tertiary processing units are configured to at least partially process at least some of the signals from the electrodes.

In one example, the one or more tertiary processing units are configured to transmit signal data at least partially indicative of the signals from the electrodes to the one or more central processing units.

In one example, the one or more central processing units are configured to receive and process the signal data.

In one example, the garment includes each of the one or more tertiary processing units on or near a different muscle group of the subject.

In one example, each array of electrodes includes a respective tertiary processing unit.

In one example, each of the one or more tertiary processing units at least partially processes signals from electrodes associated with different muscle groups of the subject.

In one example, the pose sensor includes at least one of: a position sensor configured to measure a position of at least part of the subject; a movement sensor configured to measure movement of at least part of the subject; a strain sensor incorporated into the garment and configured to measure strain of the garment; and, a pressure sensor configured to measure pressure imparted by at least part of the subject.

In one example, the one or more processing devices are configured to determine the muscle activations that contribute to at least one of: a joint position; a joint torque; a joint angle; a tissue stress; and, a tissue strain.

In one example, the system includes one or more pose templates indicative of reference poses and wherein the one or more processing devices are configured to: retrieve a pose template; compare the pose parameters to the pose template; and, using results of the comparison to determine at least one of: a pose indicator indicative of the subject pose; the activity indicator; and, the physical activity.

In one example, the pose templates are indicative of idealized poses associated with the physical activity.

In one example, the system includes one or more muscle templates indicative of reference muscle activation patterns and wherein the one or more processing devices are configured to: retrieve one or more muscle templates; compare the signals from the electrodes to the one or more muscle templates; and, using results of the comparison to determine at least one of: a muscle activity indicator indicative of muscle activation; the activity indicator; and, the physical activity.

In one example, the muscle templates are indicative of idealized muscle activation patterns associated with the physical activity.

In one example, the system includes one or more activity templates indicative of reference physical activity and wherein the one or more processing devices are configured to: retrieve an activity template; compare the subject pose and muscle activation patterns to the activity template; and, using results of the comparison to determine at least one of: the activity indicator; and, the physical activity.

In one example, the activity templates are indicative of idealized muscle activity patterns and idealized poses associated with the physical activity.

In one example, the templates are determined based on at least one of: measurements previously recorded for the subject; and, measurements recorded for one or more reference subjects.

In one example, the one or more processing devices are configured to retrieve a template at least partially based on at least one of: pose parameters; muscle activation patterns; an indication of the physical activity; user input commands; and, one or more physical characteristics of the subject.

In one example, the subject's physical characteristics are at least one of the subject's: height; weight; and, body proportions.

In one example, the one or more processing devices determine the physical characteristics based on user input commands.

In one example, the one or more processing devices are configured to determine the physical activity using at least one of: user input commands; pose parameters; and, muscle activation patterns.

In one example, the templates include: short activity duration templates; extended activity duration templates; high activity intensity templates; and, low activity intensity templates.

In one example, the one or more processing devices are configured to: determine one or more physiological parameters; compare the one or more physiological parameters to the template; and, determine the activity indicator using the results of the comparison.

In one example, the physiological parameters include measuring at least one of: lactic acid; impedance; hydration levels; sweat composition; ion composition; blood glucose; and, heart rate.

In one example, the one or more processing devices are configured to use machine learning to at least one of: identify the physical activity; classify the physical activity; select a template; generate a template; and, perform a comparison.

In one example, the comparison includes data on at least one of the subject's: joint coordinates; muscle activation patterns; performance; technique; and, pathology.

In one example, the comparison is based on at least one of: a subject fatigue; a degradation in subject performance; and, a change in risk of subject pathology.

In one example, the one or more processing devices are configured to predict future subject pathology.

In one example, the one or more processing devices are configured to derive one or more muscle activation parameters.

In one example, the one or more processing devices are configured to compare the muscle activation parameters to reference muscle activation parameters.

In one example, the one or more muscle activation parameters and reference muscle activation parameters are at least one of: tissue stresses; and, tissue strains.

In one example, the reference muscle activation parameters are at least one of: based on commonly known muscle parameters; based on subject physical characteristics; determined using machine learning; and, determined using user input commands.

A method for monitoring a physical activity performed by a subject, the method including: using a pose sensor to sense one or more pose parameters at least partially indicative of a subject pose; using a garment including a number of arrays of electrodes positioned on the garment so that when the garment is worn by the subject, the electrodes contact skin of the subject and generate electrical signals indicative of electrical potentials within respective muscles of the subject, each array of electrodes including a plurality of electrodes arranged in a grid; and, one or more processing devices determine: one or more muscle activation patterns at least partially using signals from the electrodes; the subject pose at least partially using the pose parameters; and, an activity indicator indicative of the physical activity at least partially based on the determined one or more muscle activation patterns and at least partially based on the determined subject pose.

A system for monitoring a physical activity performed by a subject, the system including: a pose sensor configured to sense one or more pose parameters at least partially indicative of a subject pose; and, one or more processing devices configured to: use the pose parameters to at least partially determine the subject pose; and, determine an activity indicator indicative of the physical activity at least partially based on the determined subject pose.

In one example, the pose parameters are indicative of at least one of: a location of one or more limbs of the subject; a location of one or more joints of the subject; and, an angle of one or more joints of the subject.

In one example, the pose sensor includes an imaging device configured to capture one or more images of the subject when performing the physical activity and wherein the one or more processing devices are configured to: receive image data indicative of the one or more images from the pose sensor; and, analyse the image data to determine the pose parameters.

In one example, the one or more processing devices analyse the image data at least in part by identifying at least one of: an outline of the subject; a silhouette of the subject; segments of the subject; and, the subject.

In one example, the pose sensor includes at least one of: a position sensor configured to measure a position of at least part of the subject; a movement sensor configured to measure movement of at least part of the subject; a strain sensor incorporation into the garment and configured to measure strain of the garment; and, a pressure sensor configured to measure pressure imparted by at least part of the subject.

In one example, the system includes one or more templates indicative of reference poses and wherein the one or more processing devices are configured to: retrieve a pose template; compare the pose parameters of the pose template; and, using the results of the comparison to determine at least one of: a pose indicator indicative of the subject pose; the activity indicator; and, the physical activity.

In one example, the pose templates are indicative of idealised poses associated with the physical activity.

A method for monitoring a physical activity performed by a subject, the method including: using a pose sensor to sense one or more pose parameters at least partially indicative of a subject pose; one or more processing devices determining: one or more muscle activation patterns at least partially using signals from the electrodes; the subject pose at least partially using the pose parameters; and, an activity indicator indicative of the physical activity at least partially based on the determined one or more muscle activation patterns and at least partially based on the determined subject pose.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which:

FIG. 1 is a schematic diagram of an example of a system for use in monitoring a physical activity performed by a subject;

FIG. 2 is a flow chart of an example of a method for use in monitoring a physical activity performed by a subject;

FIG. 3 is a schematic diagram of an example of a pose sensor for use in monitoring a subject pose;

FIG. 4 is a schematic diagram of an example of the electronics of the garment for use in monitoring the muscle activation patterns of the subject;

FIG. 5A is a schematic diagram of an example of a subject running with an unusual pose;

FIG. 5B is a schematic diagram of an example of a subject running with a normal pose;

FIG. 5C is a schematic diagram of an example of a pose sensor monitoring a subject while running;

FIG. 6 is a schematic diagram of an example of a pose sensor for use in monitoring a subject pose while running;

FIG. 7 is a schematic diagram of an example of motion capture trackers included in the garment for use in motion capture analysis;

FIG. 8A is a schematic diagram of an example of a position sensor for measuring the position of part of the subject;

FIG. 8B is a schematic diagram of an example of a movement sensor for measuring movement of the subject and/or part of the subject;

FIG. 8C is a schematic diagram of an example of a strain sensor for measuring strain in a garment worn by the subject;

FIG. 8D is a schematic diagram of an example of a pressure sensor for measuring pressure imparted by the subject and/or part of the subject;

FIG. 9 is a flow chart of an example of a process for calculating an activity indicator for the subject;

FIG. 10 is a flow chart of an example of a process for calculating the pose parameters for the subject;

FIG. 11 is a flow chart of an example of a process for determining a pose indicator and/or the physical activity the subject is performing;

FIG. 12 is a flow chart of an example of a process for determining a muscle indicator and/or the physical activity the subject is performing;

FIG. 13 is a flow chart of an example of a process for determining an activity indicator for the subject;

FIG. 14 is a flow chart of an example of a process for preparing the system prior to the subject commencing physical activity;

FIG. 15 is a schematic diagram of an example of a subject's tissue;

FIG. 16A is a schematic diagram of an example of a lactic acid sensor;

FIG. 16B is a schematic diagram of an example of a lactic acid sensor in use;

FIG. 17 is a schematic diagram of an example of locations of central processing units and tertiary processing units; and,

FIG. 18 is a schematic diagram of an example of electrical connections between central processing units and tertiary processing units.

### DETAILED DESCRIPTION OF THE PREFERRED EMBODIMENTS

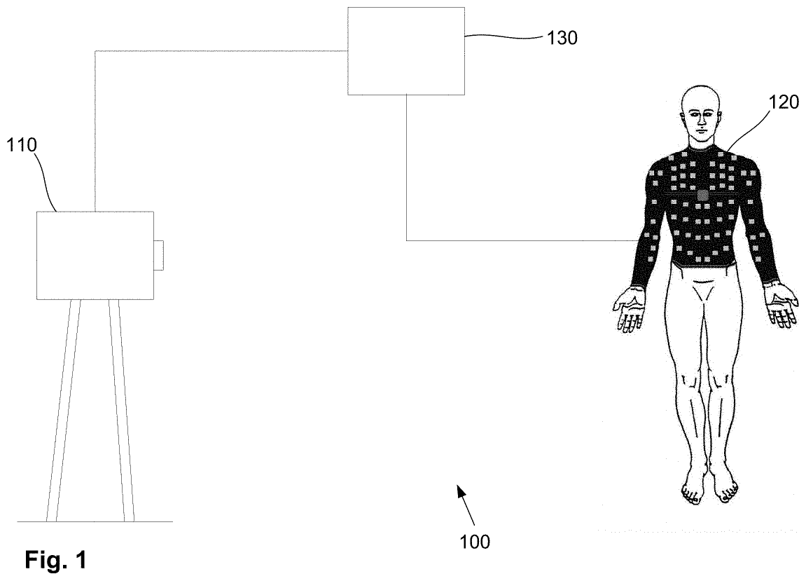

An example of a system for use in monitoring a physical activity performed by a subject will now be described with reference to FIG. 1.

The system 100 includes one or more pose sensors 110 configured so that they sense one or more pose parameters indicative of a subject pose whilst the subject is performing one or more physical activities. In this example, the pose sensor(s) 110 are in the form of an imaging camera, although other suitable arrangements can be used, as will be described in more detail below. The nature of the pose parameters will vary depending on the preferred implementation and the nature of the pose sensors, and could include information regarding an overall position of the subject, positions of individual limbs, joints, or the like.

The system 100 may include at least one garment 120, which includes a number of arrays of electrodes positioned on the garment(s) 120 so that when the garment 120 is worn by a subject in use, the electrodes contact skin of the subject and generate electrical signals indicative of electrical potentials within respective muscles of the subject. In this example, the garment(s) 120 are in the form of a shirt 120 for covering at least the torso, although other suitable arrangements can be used, such as shorts, leggings, or the like. Example garments are described in more detail in WO2016/149751 the contents of which are incorporated herein by cross reference.

In use, the pose sensor(s) 110 are in communication with one or more processing devices 130 that operate to process the one or more pose parameters and determine the subject pose. In use, the electrodes are in communication with the one or more processing devices 130 that operate to process signals and determine at least one activity indicator. The manner in which communication is achieved will vary depending on the preferred implementation and could include wired and/or wireless communication.

The one or more processing devices 130 could be of any suitable form, and could include a wearable custom or off the shelf processing device and/or a suitably programmed general purpose processing system, such as a computer system, smart phone, smartwatch, tablet or the like. Additionally or alternatively, the one or more processing devices 130 may be external to the pose sensor(s) 110 and the garment(s) 120 (as shown in FIG. 1) or integrated into the pose sensor(s) 110, the garment(s) 120 or both as well as other suitable arrangements that can be used. Furthermore, for ease of illustration the remaining description will refer to a processing device, but it will be appreciated that multiple processing devices could be used, with processing distributed between the processing devices as needed, and that reference to the singular encompasses the plural arrangement and vice versa. For example, the system could include a processing device integrated into the garment, which partially processes electrical signals from the electrodes and a separate remote processing device that calculates an activity indicator.

It will be appreciated that the system 100 may include the pose sensor(s) 110 and the processing devices 130 or may include the pose sensor(s) 110, the garment(s) 120 and the processing devices 130. The system 100 may monitor, evaluate, present feedback, provide suggestions, and/or the like regarding the subject pose without assistance from the garment(s) 120.

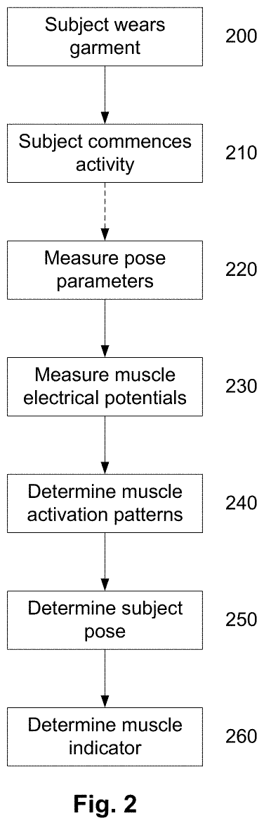

An example of a method for using the system of FIG. 1 to perform activity monitoring will now be described with reference to FIG. 2.

In this example, at step 200, the subject optionally wears the garment(s) and performs one or more physical activities at step 210. The physical activities could be of any suitable form depending on the preferred implementation or use. For example, if the subject is an athlete or sports person, the activities could include exercising, participating in a sporting event, training, or the like. Alternatively, the subject may be undergoing monitoring as part of a medical assessment, for example to assess muscle strength and/or movement patterns in individuals having muscular or neurological conditions such as myolysis, myasthenia gravis, muscular dystrophy or the like, in which case the activities could include day-to-day activities, defined sequences of movements, exercises or the like.

At step 220, the pose sensor(s) 110 measure pose parameters of the subject. The manner in which this is achieved will vary depending on the nature of the sensor. For the current example, this could include having the pose sensors generate image data indicative of a subject pose, which can then be analysed to determine the pose parameters. Alternatively, however, other pose sensors could generate other appropriate information allowing pose parameters to be determined.

Optionally, at step 230, the electrodes generate electrical signals based on electrical activity within the subject's muscles, with the signals, or data indicative of the signals, being provided to the processing devices 130 at step 240 to allow the processing devices 130 to determine a muscle activation patterns for all or parts of the respective muscles. The muscle activation patterns are indicative of the degree of electrical activity within all or respective parts of one or more muscles, such as a frequency and/or magnitude of activation, and therefor corresponds broadly to the amount of work being performed by the respective part of the muscle. Thus, signals from different pairs of electrodes within each array can be used to determine a degree of muscle activation for different parts of individual muscles or of individual muscles within a muscle group.

The processing devices 130 process pose data received from the pose sensor at step 250 to determine the subject pose, and in particular to determine pose parameters, such as an indication of a location and angles of limbs and joints of the subject, information regarding weight distribution of the subject, or the like, which are then used to determine the subject pose.

At step 260, the processing devices 130 use the subject pose and optionally the muscle activation patterns to determine at least one activity indicator indicative of the activity being performed by the subject. The indicator can be of any suitable form, and can include a number of different indicators examining different aspects of muscle activity and/or pose. For example, the system 100 can be used to determine any one or more of a joint angle indicator indicative of a position and/or angle of the subject's joints relative to the subject's corresponding limbs, a limb pose indicator indicative of a position and/or angle of limbs relative to the subject's body, a torso pose indicator indicative of posture of the subject's torso and a neck pose indicator indicative of an angle of the subject's neck relative to the subject's head and the subject's torso.

In another example, the system 100 can be used to determine any one or more of an intramuscular indicator indicative of muscle activation within a muscle, an intermuscular indicator indicative of a relative muscle activation of contralateral muscle on contralateral limbs, an efficiency indicator indicative of the relative efficiency of muscle activation of muscles when performing a task, and a muscle fatigue indicator indicative of a muscle fatigue. The indicator could include separate pose and muscle indicators, or could be a composite indicator taking into account muscle activation and pose.

In contrast to prior art techniques, in which muscle indicators are derived solely using the electrical activity of the muscles, the above described approach also takes into account the pose of the subject during the physical activity. It will be appreciated that this can be used to allow activity indicators to be derived which are more accurate and/or meaningful than muscle indicators alone. For example, the subject performing a physical activity could have an unusual pose, and yet have muscle activation patterns that are consistent with an individual performing the same activity that has a more normal pose. In this instance, basing an indicator solely on the muscle activity does not capture that the pose is unusual, which could in turn be detrimental to the subject. Similarly, where muscle activation patterns are unusual, this could be for a variety of reasons and could be due to muscle problems and/or issues with the subject's pose as they are performing the activity.

Similarly, the above described approach takes in account the muscle activation patterns of the subject while determining subject pose. It will be appreciated that this can be used to allow activity indicators to be derived which are more accurate and/or meaningful than pose indicators alone. For example, a subject performing a physical activity could have unusual muscle activation patterns, yet have a pose that is consistent with an individual performing the same physical activity that has more normal muscle activation patterns. In this instance, basing an indicator solely on the subject pose does not capture that the muscle activation patterns are unusual, which could be detrimental to the subject.

While the system 100 can derive more accurate activity indicators using pose indicators and muscle activation patterns, the system 100 can also derive activity indicators solely using pose indicators. For example, while the subject is performing a physical activity that requires symmetrical use of their muscles, such as rowing, the system 100 can determine if the subject's pose is ineffective. The system 100 may determine that the subject's pose is not symmetrical if the subject's posture and/or the subject's head is leaning to one side. Solely using pose indicators allows the system 100 to derive an activity indicator without requiring the subject to wear the garment(s) 120. This may allow the system 100 to be used for physical activities where it would impractical or impossible for the subject to the wear the garment(s) 120, such as swimming.

Accordingly, by generating an activity indicator based on the muscle activation patterns and pose information can be used to more accurately identify a source of any problems and/or corrective measures which can assist the user in addressing the issues.

In any event, it will be appreciated that the indicators can be used to assess a wide range of different aspects of performance of an activity, including for example, whether the subject is using their muscles effectively, whether the muscles are functioning as expected/required, whether the muscles are injured and/or at risk of injury, whether the subject's pose is effective, whether the pose places the subject at risk of injury and/or pathology, or the like.

A number of further features will now be described.

In one example, the one or more pose parameters are indicative of the location of one or more limbs, joints of the subject and/or the angle of one or more joints of the subject. However, it will be appreciated that other pose parameters could be used, such as a head position, a head orientation, an orientation of the one or more limbs, or the like, and that the above examples are for the purpose of illustration and are not necessarily intended to be limiting.

In one example, the pose sensor(s) 110 include an imaging device. In this example, the pose sensor(s) 110 can be configured to capture one or more images of the subject as the subject is performing the physical activity, generating image data, which can be transferred to the processing devices 130 for analysis, allowing the processing devices to determine the pose parameters. The use of an imaging device allows a wide range of different pose parameters to be easily measured, and can be deployed in a wide range of different circumstances, with a wide range of different activities. For example, this allows the pose sensor(s) 110 to be provided remotely to the subject, allowing the subject to perform the activity without being impeded by the pose sensor.

When the pose sensor(s) 110 includes an imaging device, the processing devices 130 can be configured to analyse the image data at least partially by identifying an outline of the subject, silhouette of the subject, segments of the subject and/or the subject. This could be achieved using edge detection techniques, analysing colour and/or image contrast, or the like, and this will not therefore be described in any detail. Identifying the outline or silhouette of a subject can allow the one or more processing devices 130 to conduct more accurate and/or detailed analysis of the pose, in turn allowing pose parameters to be calculated more accurately. In particular, identifying the outline or silhouette may assist in identifying the subject's pose where the analysis would otherwise mistakenly identify poses that are similar. Additionally, a more detailed analysis may assist when evaluating the quality of the subject's pose, which may be valuable for diagnostic, efficiency analysis, or the like. However, this is not essential and other analysis approaches can be used.

Identifying segments of the subject allows the processing devices 130 to associate and/or group segments belonging to the subject. Alternatively, the processing devices 130 may identify the subject first and then estimate segments of the subject, using the estimates to calculate the subject pose. These methods may assist in identifying the subject where multiple people are in view of the pose sensor(s) 110 and/or where the system 100 is monitoring multiple subjects at once to distinguish between the subjects.

In one example, the garment includes at least one marker 501 and the pose sensor(s) 110 are configured to detect the marker(s) 501. By providing marker(s), this can be used to allow the processing devices 130 to determine the subject pose with a higher degree of accuracy and/or more consistency by using the marker(s) 501 as reference point(s). The marker(s) 501 may be part of a passive optical system and/or an active optical system for capturing motion capture. Passive markers 502 reflect light and the imaging device is configured to only sample light reflected from the passive markers 502, in one example the passive markers 502 include retroreflective markers 504. Active markers 503 may be LEDs, inertial measurement units, and/or the like. The imaging device is configured to triangulate the positions of the LEDs as one LED is illuminated at a time. Alternatively, inertial measurement units positioned on the subject may assist in determining the subject pose. In one example, the garment(s) 120 include marker(s) 501, or similar, located on an exterior surface, allowing these to be captured by an imaging device, so that the imaging device can more accurately measure subject pose.

In one example, the processing devices 130 are configured to determine at least one pose parameter using motion capture techniques. The pose sensor(s) 110 capture the subject's movement and generate data indicative of the subject's movement. The processing devices 130 can determine pose parameters from the data indicative of the subject's movement. The pose sensor(s) 110 can capture the entire subject's movement simultaneously, which increases efficiency, effectiveness or the like of the system 100. It will be appreciated that such motion capture techniques can be ideally combined with the above described markers integrated into the garment to provide a high degree of accuracy, whilst allowing pose parameters to be captured for a range of different poses throughout the course of the physical activity being performed. However, it will be appreciated that other techniques for motion capture may be used, for example, non-optical systems including inertial systems, mechanical motion, magnetic systems, or the like.

In one example, the processing devices 130 can include one or more central processing units and one or more tertiary processing units. The tertiary processing units can be included in the garment near each group of muscles of the subject or each array of electrodes could include a respective of the tertiary processing unit. This allows each tertiary processing unit to only receive signals generated from a single array of electrodes, resulting in increased effectiveness and consistency of the system. The tertiary processing units can also be configured to at least partially process the signals, resulting in signal data. The tertiary processing units can be further configured to transmit the signal data to the central processing units.

A person skilled in the art would appreciate that the one or more central processing units can be included anywhere in the garment that is appropriate for the desired implementation. The central processing units could be of any suitable form, and could include a custom or off the shelf processing device and/or a suitably programmed general purpose processing system, such as a computer system, smart phone, smartwatch, tablet or the like. The central processing units can be configured to receive signal data from the tertiary processing units, combining the received signal data and/or processing the received signal data. By configuring a plurality of processors to undertake different tasks, it will reduce the processing burden on each processing unit, resulting in a lower likelihood that any processing unit will fail, increasing the reliability and durability of the system. The central processing units can also be configured to transmit data to the tertiary processing units. This may allow the central processing unit to provide instructions to the tertiary processing units and/or the electrode arrays.

For example, the system may monitor the subject using an array of electrodes on each forearm, and each array of electrodes could include one or more tertiary processing units. Each tertiary processing unit can be configured to at least partially process data solely from the array of electrodes it is connected to. This allows for the cause of software or hardware errors to more easily identifiable. If the array of electrodes on the subject's right arm malfunction, this will be apparent in the data processed by the tertiary processing unit connected to the malfunctioning array of electrodes. If the tertiary processing unit malfunctions, this will be apparent as there will be an absence of data or the data may be corrupted.

Additionally, this allows signals from each array of electrodes to be processed independently in parallel, reducing overall processing time, and allowing a real time indicator to be generated more readily.

In one example, the pose sensor(s) 110 can additionally and/or alternatively include any one or more of a position sensor, a movement sensor, a strain sensor and/or a pressure sensor, or the like. The position sensor, movement sensor, strain sensor and/or pressure sensor may be attached to the subject externally to the garment 120 and/or may be included in the garment 120.

For example, a position sensor, such as an inductive sensor and/or a proximity sensor, can be configured to determine its own position and may assist the processing devices 130 with analysis of the subject's pose by providing further information. For example, the position sensor may be included in a wearable device, such as a watch, fitness band or similar, allowing the position sensor to determine the position of the wearable device, and by extension the position of a corresponding part of the subject's body, such as the subject's wrist, or the like.

A movement sensor, such as an accelerometer, can be configured to measure a speed, direction and/or acceleration of all or part of the subject. Speed and acceleration data may assist the processing devices 130 to determine the subject pose, particularly where the subject is engaging in high speed or high intensity activity.

A strain sensor can be incorporated into the garment and used to measure strain of the garment, which can in turn be used to derive pose information. For example, stretching of arms of a garment can indicate movement of the subject's arms, allowing the processing devices 130 to determine the subject pose. This is particularly beneficial as this allows the pose and electrical signals to be measured using the garment, in turn avoiding the need for additional separate pose sensing equipment. This can also be beneficial in measuring pose where the subject pose may be partially obscured to external measuring devices, for example if the subject is squatting, which may prevent an imaging device from capturing the pose of the subject's legs. Additionally, the strain sensor can assist in determining vertebral segment movement, for example by including the strain sensor on the subject's back. During a physical activity, the strain sensor on the subject's back may measure excessive movement of certain intervertebral segments, which may result in excess stress and/or strain of tissues, which may result in spinal pain. In this example, the system 100 may inform the subject of the excessive movement, potentially allowing the subject to avoid spinal pain.

A pressure sensor can be configured to measure pressure imparted by the subject and/or a part of the subject. Pressure data may assist the processing devices 130 to determine the subject pose, where the subject pose may be difficult, inefficient and/or ineffective to measure purely from a visual means. For example, the pressure sensor may be included in one or more shoes and be configured to measure the pressure exerted by one or more of the subject's feet, or could be incorporated into an activity mat, allowing the pressure of multiple points of contact, such as hands and/or feet to be measured. The data indicative of the pressure exerted by one or more of the subject's feet may assist in determining how the subject leans, transfers their body weight or the like.

The processing devices 130 may determine the subject pose using the data provided by the pose sensor(s) 110, including an image sensor, the position sensor, movement sensor, strain sensor and/or the pressure sensor. The pose sensor(s) 110 could be used independently, and/or could be used in conjunction. This could be performed to improve accuracy of measurements, allowing measurements from multiple sensors to be combined to ascertain a pose, or to provide back-up functionality, for example to supplement an image sensor using readings for other sensors where line of sight is lost or where quality of line of sight is inconsistent. For example, if the subject is engaging in physical activity where other individuals may impede the line of sight between the pose sensor(s) 110 and the subject, the processing devices 130 may still be able to determine the subject pose using the data provided by other pose sensors, such as the position sensor, the movement sensor, the strain sensor and/or the pressure sensor.

In one example, the processing devices 130 are configured to determine the muscle activations that contribute to joint position, joint torque, joint angle, tissue stress, tissue strain and/or the like. Determining these characteristics allows the system 100 to determine more efficient, effective and consistent activity indicators as the processing devices 130 can make the determination using more information and a larger variety of information. It will be appreciated that tissue includes biological tissue (such as ligaments, muscles, tendons, bones, and/or the like) and/or non-biological tissue (such as hip and/or knee replacements, 3D printed bones, 3D printed tissues, disc replacements, spinal fusions, and/or the like).

The exact manner in which activity and/or pose and muscle indicators are derived will vary depending on the preferred implementation. In one example, the system 100 uses one or more templates to analyse the pose and/or muscle activity. The templates can include pose, muscle activation or activity templates, and are typically indicative of respective “idealised” behaviour when performing a given physical activity. Thus, the pose template could be indicative of an “idealised” subject pose when performing a given physical activity. Similarly, the muscle templates can be indicative of “idealised” reference muscle activation patterns, whilst activity templates are indicative of “idealised” combinations of muscle activation patterns and subject poses.

The templates may be based on physical activity the subject has previously performed, could be determined based on one or more other individuals performing that physical activity, could be determined using a simulated person performing that physical activity, or the like.

Thus, in one example, the templates are determined based on measurements previously recorded for the subject and/or measurements recorded for one or more reference subjects. Measurements previously recorded for the subject, or historical measurements, provide data indicative of how the subject will perform the physical activity in the future. Determining the template based on previously recorded measurements can improve the efficiency and effectiveness of the system 100.

Alternatively, measurements recorded for reference subject(s) can provide an indication of how a typical subject will perform a physical activity. Determining the template based on measurements recorded for a reference subject can improve the efficiency and effectiveness of the system 100, for example, where measurements previously recorded for the subject are unavailable, unreliable and/or the like, or to account for the fact that a subject may have an initial non-ideal muscle activation or pose.

When the reference pose is based on reference subjects, these could be other individuals having similar physical characteristics, similar physical abilities or disabilities, similar levels of expertise or training, or the like. Thus, in one example, the processing devices 130 are configured to determine more or more physical characteristics of the subject and retrieve the template based on the one or more physical characteristics, so that the retrieved template is derived from reference subjects having similar physical characteristics. This avoids measurements for a subject being compared to templates derived from reference subjects having substantially different physical characteristics, which could result in the processing devices 130 being unable to accurately to determine a relevant indicator. Additionally, the processing devices 130 will be able to more efficiently determine a template for an average and/or reference subject as providing additional information will narrow the number of relevant templates. The subject's physical characteristics could include height, weight, body proportions and/or the like.

In one example, the processing devices 130 determine physical characteristics based on user input commands. This allows the user to input the physical characteristics of the subject without the need for the processing devices 130 to determine them. This removes a task required from the processing devices 130 to complete, improving efficiency, and the user can input all relevant information accurately, improving effectiveness. However, it will be appreciated that this is not necessarily essential, and physical characteristics can be determined using other approaches, such as analysing captured images of the subject, and using these to derive the physical characteristics.

In one example, the processing devices 130 are configured to determine a physical activity being performed by the subject, retrieve templates based on the physical activity and determine the activity indicator at least in part using the templates. In this example, the processing devices 130 can be configured to determine the physical activity using user input commands, pose parameters and/or muscle activation patterns. Depending on the application, the subject may wish to choose a specific physical activity for the system 100 to compare to the subject's physical activity.

The processing devices 130 are typically configured to retrieve a template, compare the pose parameters and/or muscle activity to the template and use results of the comparison for further assessment. Thus, pose parameters could be compared to a pose template, muscle activation patterns could be compared to muscle templates and muscle activation patterns and pose parameters could be compared to activity templates.

The processing devices 130 may use results of the comparison to determine an indicator. For example, pose templates could be used to determine a pose indicator indicative of a subject pose, and in particular to inform the subject on a specific aspect of their pose, such as parts of their pose that are ideal or not ideal. Thus, pose templates can be indicative of idealised poses and for a given physical activity there are multiple different poses a subject can have. By comparing the subject's pose parameters to idealised pose template(s), deficiencies and/or inefficiencies in the subject's pose can be more easily identified. The subject can then more readily improve their technique or reduce their risk of pathology.

Similarly, muscle templates could be used to determine a muscle indicator indicative of a muscle activity, and in particular to inform the subject on how their muscle activation deviates from an idealised activity. In one example, the muscle templates are indicative of idealised muscle activation patterns. Thus, for a given physical activity there are a variety of muscle activation patterns a subject can have. By comparing the signals from the electrodes to an idealised muscle template, deficiencies and/or inefficiencies in the subject's muscle activation patterns can be more easily identified. The subject can then more readily improve their technique or reduce their risk of pathology. Thus, the muscle indicator can be indicative of the how the subject's muscles are operating, including the efficiency, effectiveness and/or potential pathology of the subject.

In one example, the activity templates are indicative of idealised physical activity. By comparing the subject pose and muscle activation patterns to an idealised activity template, deficiencies and/or inefficiencies in the subject's pose and muscle activation patterns can be more easily identified, again allowing the subject to more readily improve their technique or reduce their risk of pathology.

In one example, the templates include short activity duration templates, extended activity duration templates, high activity intensity templates and/or low activity intensity templates. It will be appreciated that other templates may be included or any combination of the templates previously discussed. A subject's pose and their muscle activation patterns may vary when performing the same activity at different intensities or different durations. Including templates that take these variations into account allows for more efficient and effective templates. Additionally, when making comparisons to measured data and these templates, it allows the processing devices 130 to determine more relevant indicators to the physical activity being performed.

In one example, the processing devices 130 can be configured to determine one or more physiological parameters, compare the physiological parameters to the selected template and determine the activity indicator using the results of the comparison. Depending on the physical activity being performed, the system 100 may be able to determine a more accurate and/or useful activity indicator if supplied with additional physiological parameters (such as measuring lactic acid, impedance, hydration levels, sweat composition, ion composition, blood glucose, heart rate and/or the like). For example, if the subject is running for an extended period they may build lactic acid in their muscles. Should the lactic acid measurement be too high, then the system 100 may advise the subject to rest even when the subject's technique is healthy and/or ideal.

In one example, the processing devices 130 can be configured to use machine learning to select a template, generate a template and/or perform a comparison. As a subject continues to use the system 100, the processing devices 130 can use machine learning to select or generate a template that is more appropriate, allowing the use of machine learning to be used to improve efficiency and effectiveness by developing learning models over time. The processing devices 130 can use machine learning to create templates specific to the user, which allows the created templates to be more effective comparisons to the user and automatically tune templates to changes in the subject over time. Machine learning can take into account details specific to the user, such as physical characteristics, age, pathology, technique, endurance and/or the like, to perform comparisons more relevant to the subject in the present and future.

For example, the machine learning technique can involve capturing multiple pose parameters and/or muscle activation patterns for a given physical activity, either from the subject, or multiple reference subjects, and using these to derive idealised templates. This can be performed using appropriate techniques, such as decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known, these will not be described in any further detail.

Additionally or alternatively, the system 100 can derive the idealised templates. The system 100 could derive idealised templates using a collection of templates from healthy individuals where the system 100 may receive the collection of templates from an external source or derive the collection of templates by assessing healthy individuals, for example using the above described measurement process to obtain pose and/or muscle activation measurements for healthy individual performing activities. Each template from the collection of templates could include activity indicators, pose parameters and/or muscle activation patterns of a healthy individual as they performed a physical activity. The system 100 can then analyse the activity indicators, pose parameters and/or muscle activation patterns from the collection of templates to derive an idealised template for the physical activity.

For example, the system 100 could receive a collection of templates that included activity indicators, pose parameters and muscle activation patterns of healthy individuals as they row. After analysing the collection of templates, the system 100 could derive an idealised template including optimised values for activity indicators, pose parameters and muscle activation patterns. The idealised template could include that a healthy individual, while rowing, should maintain symmetry over the left and right sides of their body, as they activate their muscles, position their joints and their overall posture. Additionally, the idealised template could be reviewed by an expert to ensure that the optimised values are accurate.

In one example, this process involves generating a computational model for each activity, using a generic model, which is then trained using pose parameters and/or muscle activation patterns from previous measurements for the subject, or measurements for reference individuals. Pose parameters and/or muscle activation patterns measured from the subject can then be applied to the computational model, to thereby generate an indicator, such as a pose indicator, muscle indicator or activity indicator, depending on the preferred implementation. For example, the computational model for squats may be created by taking a sample of individuals who are known to demonstrate a healthy and/or ideal technique. The healthy and/or ideal technique may be where the lower limb extension is driven by the most efficient weight contributions from each major muscle group (such as gluteals, quadriceps, hamstrings, calfs and/or the like) to produce minimal shearing forces at the knee. As a further example, the subject may be identified as having a non-healthy and/or non-ideal technique if their quadriceps contribution is too large, which may cause the patella to tighten against the femur which may result in pain or anteriorly translate the tibia causing sheering of the meniscus.

Alternatively, the computational model may be generated solely using computational analysis without taking measurements from reference individuals. The computational model may be more finely tuned without being restricted by the limitations and/or inconsistency of a reference individual. For example, this allows the subject seeking a high standard of physical activity to be compared to a model that may not normally be found in an individual. For example, a completely idealised computational model for a squat may be generated using computational analysis calculating the different shear, rotational, lateral forces, and/or the like.

In one example, the comparison process takes into account data on the subject's performance, technique, joint coordinates, muscle activation patterns and/or pathology. When the processing devices 130 perform a comparison, they are comparing the subject's data to a template and determining differences. These differences can indicate where a subject could improve their performance, technique and/or show the impact of pathology on the subject. For example, the processing devices 130 could compare the subject performing a chin-up to a template for performing a chin-up. The comparison may show that the subject does not raise their chin to the correct position, an error that may not be perceptible to the naked eye. By showing this error to the subject, they may improve their technique and result in a more efficient and/or more effective training regime over time.

In one example, the comparison is based on a subject fatigue, degradation in subject performed and/or a change in the risk of subject pathology. The processing devices 130 may perform a comparison on a subject over more than a single instance resulting in a comparison that may vary over time. When performing a physical activity, the subject may fatigue over time, resulting in a change in their pose or muscle activation patterns. By comparing the changes in a subject's physical activity over time, the processing devices 130 may determine indicators that are more relevant to the physical activity. For example, as a football player fatigues over the course of a match, their technique made degrade by the end of the match shown by an increased risk of pathology and/or a degradation of performance. By monitoring these factors, the processing devices 130 is more likely to determine that a change in a subject's technique.

In one example, the processing devices 130 are configured to predict future subject pathology. Using present comparisons, historical data, machine learning and/or the like, the system 100 may be able to predict when a subject could suffer future pathology based on their current technique and/or method of conducting a physical activity. This information can be presented to the subject, potentially also with suggestions in how to alter their performance of the physical activity, to prevent the subject from suffering future pathology.

The processing devices 130 can be configured to derive one or more muscle activation parameters. If the processing devices 130 are unable to determine a suitable template, they are configured to derive muscle activation parameters from data provided from the garment(s) 120 and/or the pose sensor(s) 110. The muscle activation parameters are indicative of the physical activity of the subject.

In one example, the processing devices 130 can be configured to compare the muscle activation parameters to reference muscle activation parameters. Reference muscle activation parameters are indicative of a subject's physical activity when performing a given physical activity. By comparing the muscle activation parameters to reference muscle activation parameters, analysis on the subject's physical activity may be possible where comparing to templates is impossible, impractical, inefficient, ineffective or the like.

The muscle activation parameters and reference muscle activation parameters could include tissue stresses and/or tissue strains. The processing devices 130 can compare the tissue stresses and/or tissue strains measured from the subject to reference tissue stresses and/or tissue strains. From this comparison, the processing devices 130 may be able to determine an indicator for the subject's physical activity.

In one example, the reference muscle activations parameters are based on commonly known muscle parameters, based on subject physical characteristics, determined using computational analysis, determined using machine learning and/or determined using user input commands. Where little additional data is supplied to the system 100, the processing devices 130 may use commonly known muscle parameters, such as maximum tissue strains and tissue stresses that occur during a physical activity. The processing devices 130 may measure the subject's physical characteristics and use those characteristics to determine the reference muscle activation parameters. The processing devices 130 may perform computational analysis to create the reference muscle activation parameters. The processing devices 130 may use machine learning to create the reference muscle activation parameters, for current or future use. Alternatively, a user may input reference muscle activation parameters. When using any of these methods, it allows the processing devices 130 to flexibly determine an indicator of physical activity where there is insufficient information and/or data to determine a template.

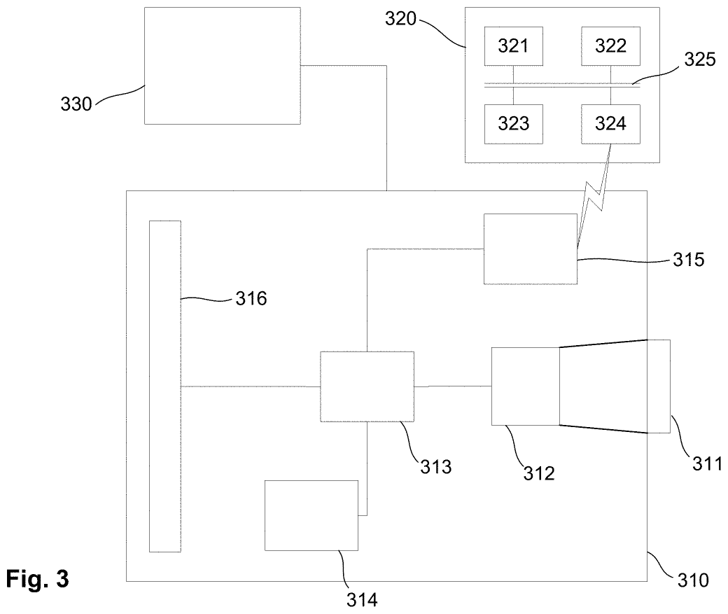

An example of a pose sensor 110 will now be described with reference to FIG. 3.

The pose sensor(s) 110 includes a pose sensor body 310, a host device 320 and power supply, such as a battery 330. The pose sensor body 310 includes a lens 311, the imaging device 312, a microprocessor 313, memory 314, an external interface 315 and an external connector 316. The imaging device 312, microprocessor 313, memory 314, external interface 315 and external connector 316 are in communication with each other.

The lens 311 focuses light so that the imaging device 312 may create image data indicative of one or more images. The memory 314 may permanently or temporarily store image data and/or pose parameter data. The external interface 315 is connected to a host device 320 and allows for the transmission of data to the host device 320. The external interface 315 and host device 320 may be electrically or wirelessly connected by any means known in the art allowing the transfer of data to the host device 320. The external connector 316 may connect the pose sensor 110 to the garment(s) 120, processing devices 130 and/or anything else required by the implementation of the invention.

The host device 320 includes a microprocessor 321, a memory 322, an input/output device 323, such as a keyboard and/or display, and an external interface 324, interconnected via a bus 325 as shown. In this example the external interface 324 can be utilised for connecting the host device 320 to peripheral devices, such as the measuring device 410, as well as other devices, such as communications networks, databases, other storage devices, or the like. Although a single external interface 325 is shown, this is for the purpose of example only, and in practice multiple interfaces using various methods (eg. Ethernet, serial, USB, wireless or the like) may be provided.

In use, the microprocessor 321 executes instructions in the form of applications software stored in the memory 322 to allow communication with the measuring device 410, for example to control operation of the measuring device 320, to receive outputs therefrom, and to at least partially process outputs to create and display representations of one or more indicators.

Accordingly, it will be appreciated that the host devices 320 may be formed from any suitable processing system, such as a suitably programmed PC, Internet terminal, lap-top, or hand-held PC, and in one preferred example is either a tablet, or smart phone, or the like. However, it will also be understood that the host device 320 can be any electronic processing device such as a microprocessor, microchip processor, logic gate configuration, firmware optionally associated with implementing logic such as an FPGA (Field Programmable Gate Array), or any other electronic device, system or arrangement.

The battery 330 is electrically connected to the pose sensor body 310, the imaging device 312, the microprocessor 313, memory 314, external interface 315 and external connector 316. The battery 330 may be external to the pose sensor body 310 (as shown in FIG. 3) or internal to the pose sensor body 310 (not shown).

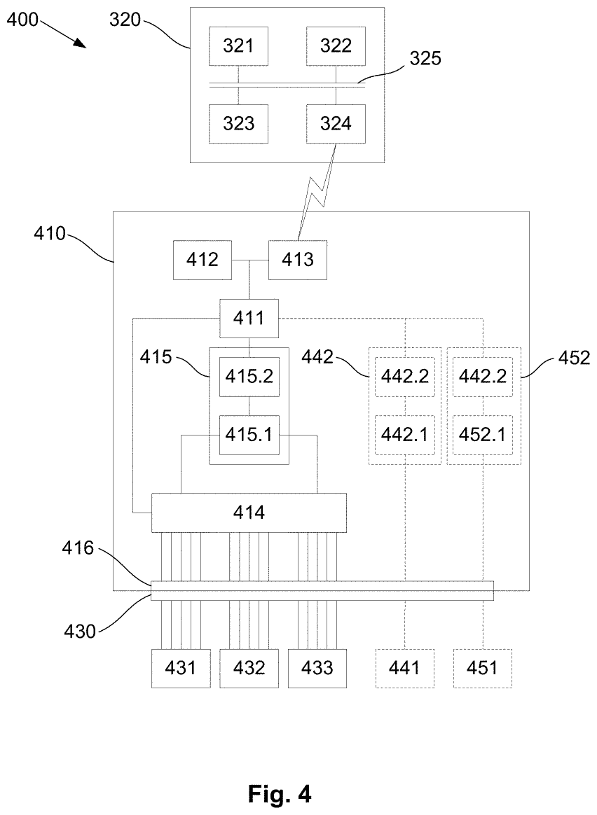

An example of a garment 120 will be described with reference to FIG. 4.

The garment 120 includes a measuring device 410, which is adapted to be coupled to the garment and worn by the user, and a host device 320, which is in communication with the measuring device, to allow indicators to be displayed thereon.

In this example, the measuring device 410 includes a microprocessor 411, coupled to a memory 412 and an external interface 413, such as a wireless communications interface for communicating with the host device 320.

The measuring device 410 includes a switching device 414, such as a dual band multiplexer, which is coupled to respective electrode arrays 431, 432, 433, via a port 416 and corresponding connector 330 provided on the garment. Although three electrode arrays are shown, this is for the purpose of illustration only, and in practice any number of electrode arrays maybe provided, depending on the preferred implementation.

The switching device 414 is also coupled to a voltage sensor 415, allowing signals from respective pairs of electrodes within the arrays to be provided thereto. The voltage sensor typically includes a differential amplifier 415.1, for amplifying a potential difference across the pair of electrodes and an analogue-to-digital convertor (ADC) 415.2 for digitising the resulting potential difference signal. An optional filter (not shown) is also provided for filtering the signals for example using a bandpass filter, to remove any signal components from other sources, such as noise in the leads, or other biological signals, such as ECG signals or the like. Accordingly, in use, the voltage sensor 415 amplifies the potential difference between the electrodes in the respective pair, and generates an analogue voltage signal, which is then filtered and digitised before being provided to the processor 411 for analysis.

In use, the processor 411 controls the switching device 414 to connect the differential amplifier to respective pairs of electrodes within a given electrode array 431, 432, 433, based on a defined measurement protocol. The measurement protocol typically defines a sequence of pairs of electrodes from which measurements should be taken, and may be stored in the memory 412 and selected depending on the desired outcome, as will be described in more detail below.

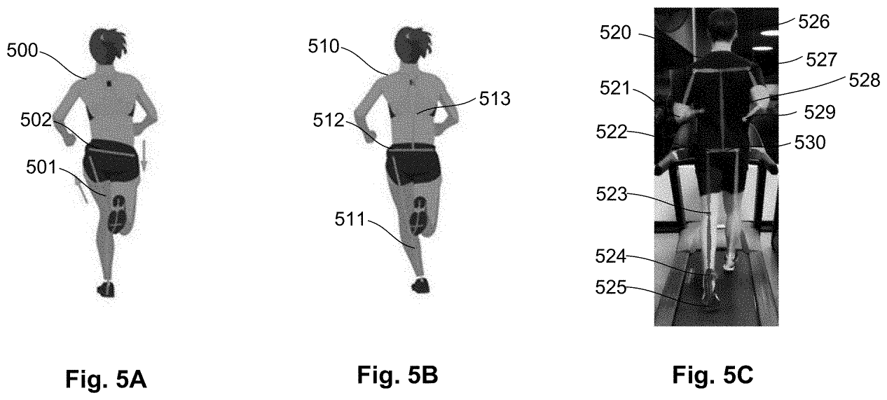

An example of a subject with unusual and normal pose will be described with reference to FIGS. 5A, 5B and 5C.

FIG. 5A shows an example of an individual 500 that is running with an uneven and/or unusual posture. This can be seen by the kink in their supporting leg 501 and the slant in their waist 502. Conversely, FIG. 5B shows an example of an individual 510 that is running with an even and/or normal posture. This can be seen by their straight leg 511, flat waist 512 and straight spine 513. The individual 510 may be included in the system 100 as an example of healthy and/or ideal method of running.

In this example, the system 100 may compare the physical activity of the individual 500 to the physical activity of the individual 510, determining similarities and differences between the two. In this example, the system 100 may then indicate to the individual 500 that their method of running may result in injury or pathology and potentially suggest methods for correcting their technique.

FIG. 5C shows an example of the system 100 monitoring an individual 520 and identifying the individual 520 pose. In this example, the system 100 identifies an elbow 521, hip 522, knee 523, ankle 524, toe 525, ear 526, shoulder 527, spine 528, wrist 529 and centre pelvis 530. It will be appreciated that any combination of these features could be identified including others of a similar kind. The system 100 may use the identified features to determine the physical activity the individual 520 is performing.

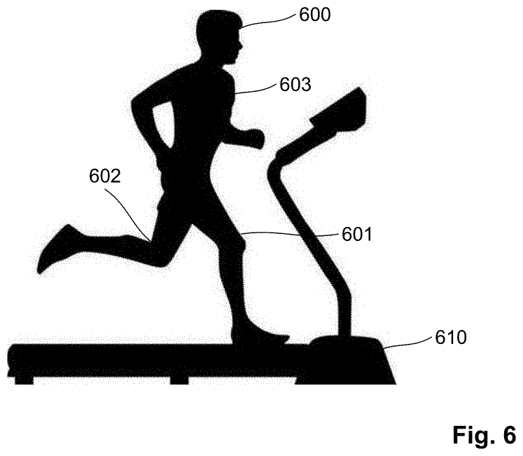

An example of a pose sensor 110 monitoring a subject running will be described with reference to FIG. 6.

FIG. 6 shows an example of an individual 600 that is running on a treadmill 610. It will be appreciated that the pose sensor 110 may also monitor the individual 600 while not running on a treadmill. In this example, the system 100 may monitor joint positions 601, joint angles 602 and/or the orientation and/or angle of the subject's chest 603 and may analyse these parameters to determine if the individual 600 has a healthy and/or ideal technique while running. Additionally, the system 100 may monitor joint positions 601 and/or joint angles 602 that are not shown in FIG. 6 (such as elbows, ankles, wrists, hip, neck, and/or the like).

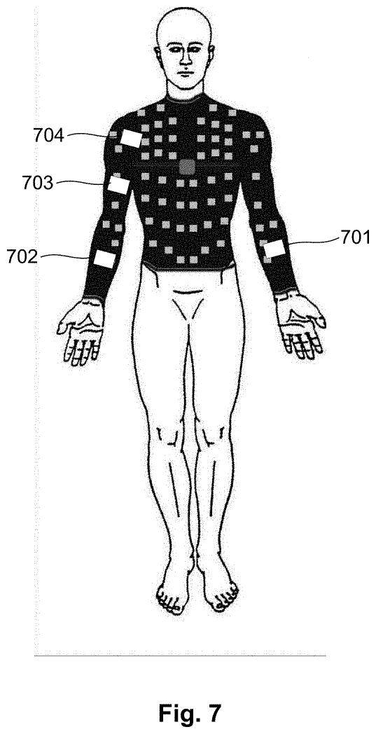

An example of motion capture arrangement will be described with reference to FIG. 7.

The garment 120 includes at least one marker 701, which is configured to be detected by the pose sensor 110. The marker 701 is a generic marker and may be of any form that allows the processing devices 130 to perform motion capture analysis. The garment 120 may also include a passive marker 702, an active marker 703 and/or a retroreflective marker 704.

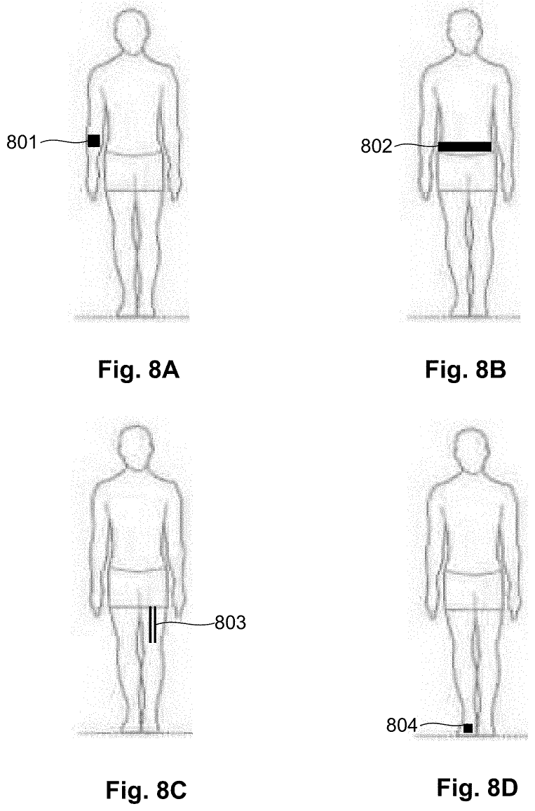

An example of pose sensor(s) 110 including wearable sensors will now be described with reference to FIGS. 8A, 8B, 8C and 8D.

FIG. 8A shows an example of a position sensor 801 located on the subject's arm. FIG. 8B shows an example of a movement sensor 802 located on the subject's stomach. FIG. 8C shows an example of a strain sensor 803 located on the subject's leg. FIG. 8D shows an example of a pressure sensor located on the subject's foot. It will be appreciated that any of these sensors may be located on any part of the subject's body, as appropriate for the implementation. Alternatively, the sensors may be included in the garment 120 (not shown).

Examples of the processes for monitoring muscle activity will now be described in further detail. For the purpose of these examples it is assumed that the host device 320 is a smart phone, tablet, smartwatch or other similar computing device that executes a software application that allows for communication with one or more measuring devices 410, each of which is associated with a respective garment.

If the host device 320 is a computing device that includes an imaging device (such as a smart phone, tablet, smartwatch, laptop, and/or the like) then the pose sensor(s) 110 and host device may be part of the same device. In this example, the computing device that includes an imaging device may monitor the subject performing a physical activity, capture image data, identify the subject pose, determine the muscle activation patterns, and/or the like in a single device. This may be convenient for the subject, as a smart phone set up to observe the subject running on a treadmill may be all that is necessary for the system 100 to function.

Alternatively, the host device 320 and pose sensor(s) 110 may be separate devices. In this example, the pose sensor(s) 110, which may be an imaging device (such as a camera), may monitor a sporting event while analysis of the sporting event is processed and/or displayed on the host device 320 (such as a smart phone). This may assist in providing more accurate data as a dedicated camera may capture more accurate and/or detailed image data than a smart phone and may allow for more subjects to be monitored simultaneously. Additionally, the processing devices 130 that process the analysis of the sporting event may be part of a cloud based processing device instead of part of the host device 320. This may allow the system 100 to monitor more subjects at once by making use of more processing power and may prevent the host device 320 from being excessively depleted in battery.

However, it will be appreciated that the above described configuration assumed for the purpose of the following examples is not essential, and numerous other configurations may be used. It will also be appreciated that the partitioning of functionality between the host devices 320, and the measuring devices 410 may vary. For example, the operation of the measuring device 410 could be controlled by a user via a user interface displayed on the measuring device, allowing the measurement process to be performed, with data indicative of measured signals being pushed to the host device, thereby allowing measurements to be performed without requiring the host device. It will also be appreciated that the host device could be connected to one or more other processing systems, such as part of a cloud or other distributed architecture.

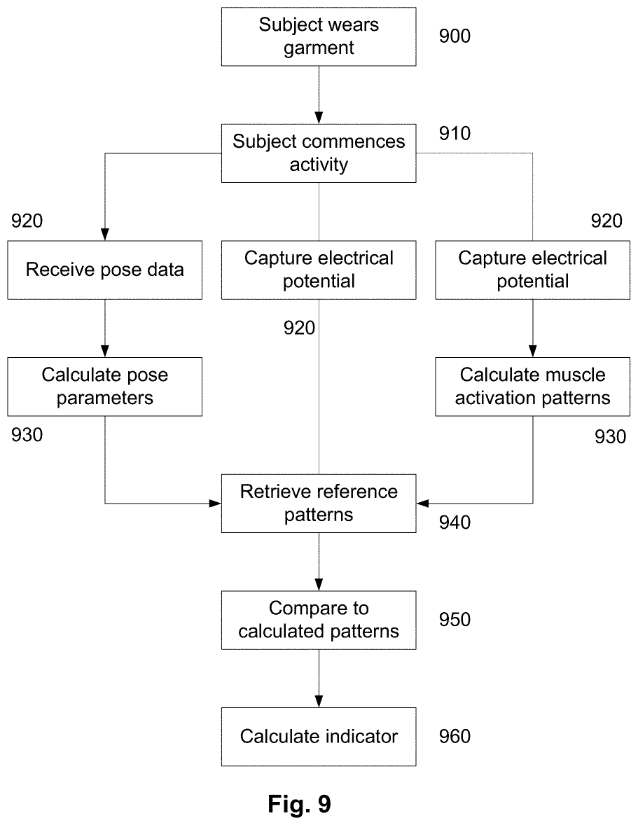

An example of a process for calculating an activity indicator will be described with reference to FIG. 9.

At step 900 the subject wears the garment and commences a physical activity at step 910. At step 920 the pose sensor 110 receives pose data and the electrodes 121 capture electrical potential created by the subject as they perform the physical activity. At step 930 the processing devices 130 calculate pose parameters and muscle activation parameters. Optionally, at step 920 the system 100 may measure other data that may assist in calculating pose parameters and/or muscle activation parameters (such as measuring lactic acid, respiratory rate, hydration levels, impedance, sweat composition, ion composition, blood glucose, heart rate, and/or the like). It will be appreciated that the actions performed at steps 920 and step 930 may occur simultaneously (as shown in FIG. 9), sequentially, progressively or in parts.

At step 940 the processing devices 130 retrieve references, including reference muscle activation patterns and reference pose parameters that are appropriate to the physical activity being performed. At step 950 the processing devices 130 compare the measured muscle activation patterns and pose parameters to the reference muscle activation patterns and reference pose parameters, determining any similarities and differences between them. At step 960 the processing devices 130 calculate an activity indicator indicative of the physical activity using the comparison.

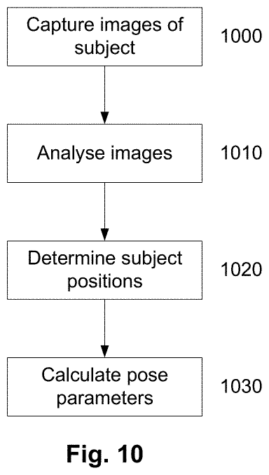

An example of a process for calculating pose parameters will be described with reference to FIG. 10.

At step 1000 the pose sensor 110 captures images of the subject performing a physical activity and converts the images into image data indicative of images of the subject performing a physical activity. At step 1010 the processing devices 130 analyse the image data, for example by using image processing techniques to identify an outline of the subject (such as identifying 2D keypoints, determining 3D poses that map to the identified 2D keypoints and determining which 3D pose corresponds to the physical activity being performed), segments of the subject and/or the subject, and hence a subject position at step 1020. At step 1030 the processing devices 130 calculate pose parameters using the subject's position determined in step 1020, for example, using the outline of the subject to measure joint angles and positions, which can be achieved using a geometric analysis.

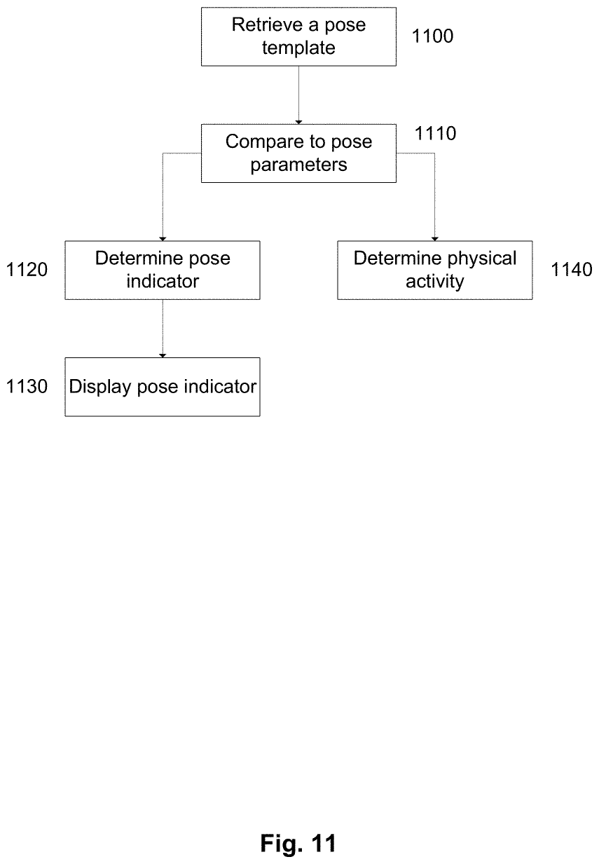

An example of a process for determining a pose indicator or physical activity will now be described with reference to FIG. 11.

At step 1100, the processing devices 130 retrieve a pose template from the system. At step 1110, the processing devices 130 compare the pose template to pose parameters to identify similarities and differences. For example, this can involve comparing joint positions and joint angles measured for the subject to idealised joint angles/positions, to identify any deviations between the measured pose parameters and idealised pose parameters embodied in the pose template.

At step 1120, the processing devices 130 determine a pose indicator, which can then be displayed at step 1130. In this regard, the pose indicator can be used to indicate deviations between the measured and idealised pose parameters, and could be of any form, including a graphical representation showing differences between the measured subject pose and an idealised pose, or could be a numerical score highlighting a degree of similarity or differences between the measured subject pose and an idealised pose. The indicator could be a single overall indicator, or could include individual scores or representations for each of a range of different aspects of pose, such as a respective score for each limb and/or joint.

Alternatively, at step 1140, the processing devices 130 could use results of the comparison to determine the physical activity being performed. For example, pose templates could be provided for a range of different activities, with a comparison being performed between the pose parameters and each of the pose templates, so that a best match is used to identify the activity being performed.

Once the activity has been determined, this information can then be used to retrieve an activity template that may be compared to one or more muscle activation patterns.

It will be appreciated that the actions performed at step 1120 may occur simultaneously (as shown in FIG. 11), sequentially, progressively or in parts.

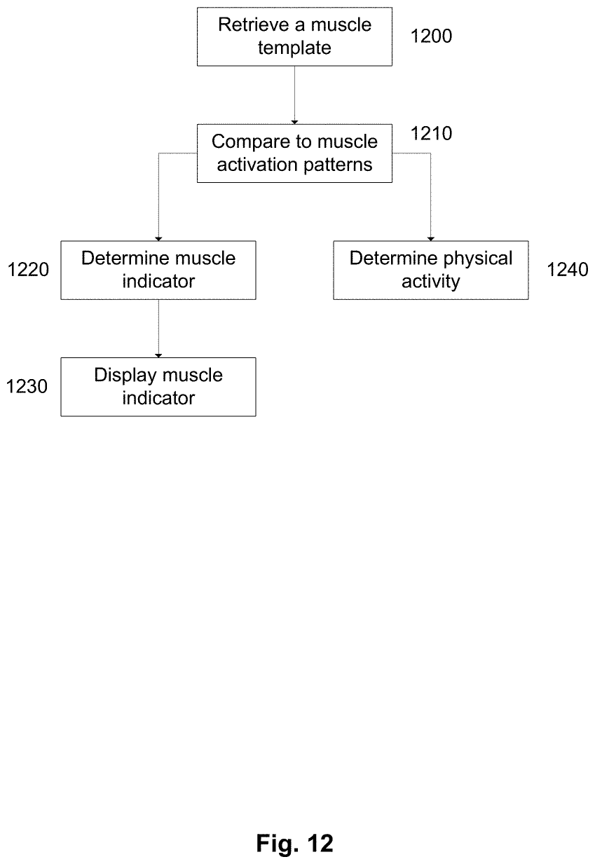

An example of a process for determining a muscle indicator or physical activity will now be described with reference to FIG. 12.

At step 1200, the processing devices 130 retrieve a muscle template from the system. At step 1210, the processing devices 130 compare the muscle template to muscle activation patterns to identify similarities and differences. For example, this can involve comparing tissue measuring frequency, tissue amplitude, tissue innervation zones, tissue strains and/or tissue stresses measured for the subject to idealised parameters, to identify any deviations between the measured muscle activation patterns and idealised muscle activation patterns embodied in the muscle template.

At step 1220, the processing devices 130 determine a muscle indicator, which can then be displayed at step 1230. In this regard, the muscle indicator can be used to indicate deviations between the measured and idealised muscle parameters, and could be of any form, including a graphical representation showing differences between the measured muscle activation patterns and idealised muscle activation patterns, or could be a numerical score highlighting a degree of similarity or differences between the measured muscle activation patterns and idealised muscle activation patterns. The indicator could be a single overall indicator, or could include individual scores or representations for each of a range of different aspects of muscle activation patterns, such as a respective score for each muscle and/or group of muscles.

Alternatively, at step 1240, the processing devices 130 could use results of the comparison to determine the physical activity being performed. For example, muscle templates could be provided for a range of different activities, with a comparison being performed between the muscle activation patterns and each of the muscle templates, so that a best match is used to identify the activity being performed.

Once the activity has been determined, this information can then be used to retrieve an activity template that may be compared to the subject pose.

It will be appreciated that the actions performed at step 1220 may occur simultaneously (as shown in FIG. 12), sequentially, progressively or in parts.

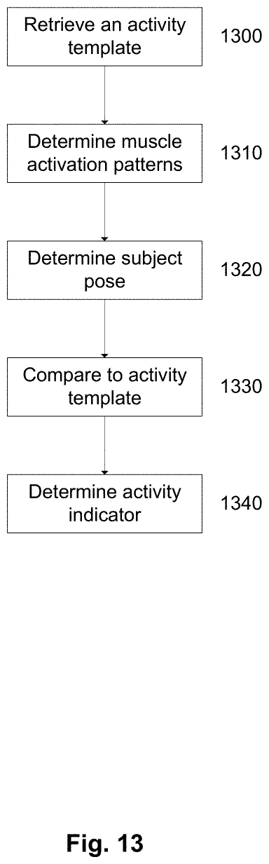

An example of a process for determining an activity indicator will now be described with reference to FIG. 13.

At step 1300 the processing devices 130 retrieve an activity template. This can be performed based on user input commands, for example indicating the activity being performed, or could be performed based on an analysis of muscle or pose parameters, as described above.

At step 1310 the processing devices 130 determine the muscle activation patterns of the subject as they perform a physical activity. At step 1320 the processing devices 130 determine the pose of the subject as they perform a physical activity. It will be appreciated that the actions performed at steps 1310 and step 1320 may occur sequentially (as shown in FIG. 11), simultaneously, progressively or in parts. At step 1330 the processing devices 130 compare the subject pose and muscle activation patterns to the activity template. At step 1340 the processing devices 130 determine an activity indicator indicative of the physical activity the subject has performed using the comparison from step 1330.

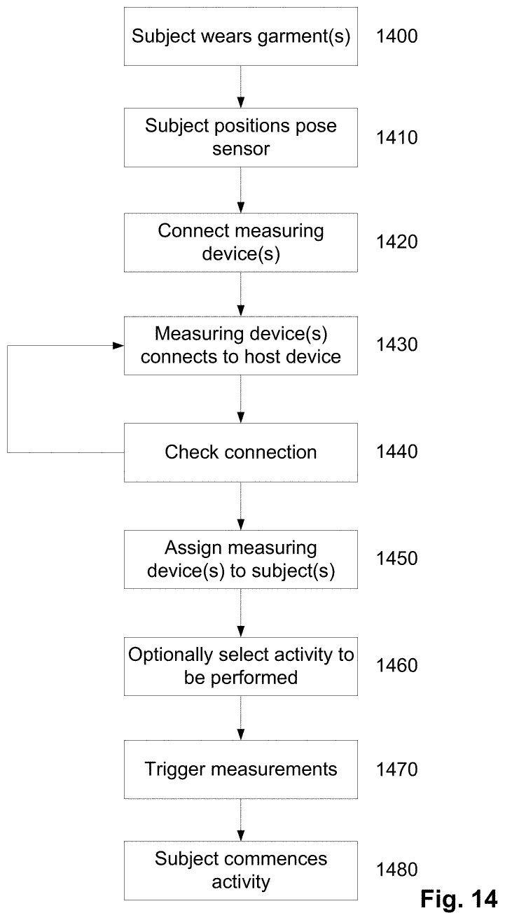

An example of a process for the subject commencing physical activity will be described with reference to FIG. 14.

At step 1400 the subject wears the garment(s) 120 and positions the pose sensor 110 at step 1410. At step 1420 the measuring devices are connected so that the garment(s) 120 and the pose sensor 110 are in communication with each other and the processing devices 130. At step 1430 the measuring devices are connected to the host device 320 and that connection is checked in step 1440. If there are any errors with that connection the user and/or subject is directed to return to step 1430 to fix the connection or reconnect the measuring devices to the host device. At step 1450 the processing devices 130 assign the measuring devices to the subject or subjects. At step 1460 the user and/or subject may optionally select an activity that is being performed, this may not occur depending on the implementation of the invention. At step 1470 the processing devices 130 take initial measurements prior to the subject commencing activity, these measurements may be for calibration purposes or confirming the equipment is functioning as intended. At step 1480 the subject commences a physical activity.

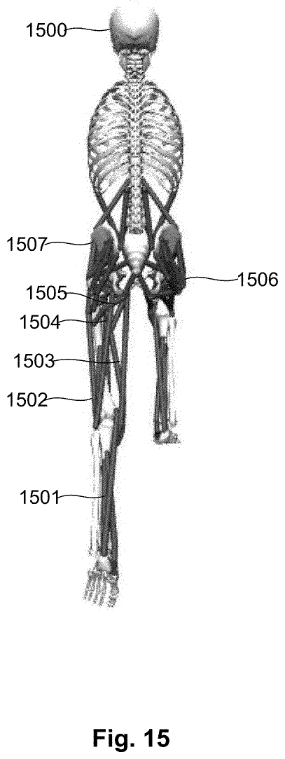

An example of the tissues analysed by the system 100 will be described with reference to FIG. 15.

The system 100 may analyse groups of tissues when determining an activity indicator for an individual 1500. These groups of tissues may be gastrocnemius muscles 1501, tensor fasciae latae muscles 1502, semimembranosus muscles 1503, biceps femoris muscles 1504, adductor magnus muscles 1505, gluteus maximus muscles 1506, gluteus medius muscles 1507, and/or the like. The system 100 may determine the stresses, strains, torques and/or similar forces in tissue groups to determine an activity indicator.

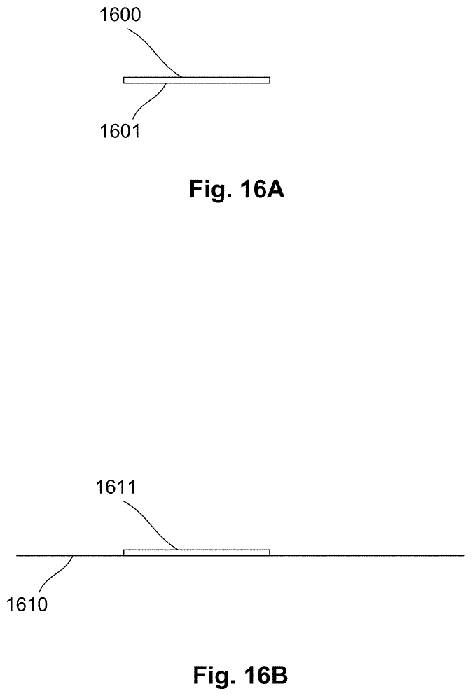

An example of a lactic acid sensor will be described with reference to FIGS. 16A and 16B.

FIG. 16A shows an example of a lactic acid sensor including an electrode 1600 and conductive ink 1601 (such as Melton JR-DEV, HPS-FH32 and/or the like). FIG. 16B shows a lactic acid sensor 1611 in use by being placed against an individual's skin 1610. The lactic acid sensor generates data indicative of the lactic acid level of the individual 1610. The data may assist the system 100 in determining an activity indicator for the individual 1610, and it will be appreciated that a sensor of this form could be incorporated into a garment similar to that described above.

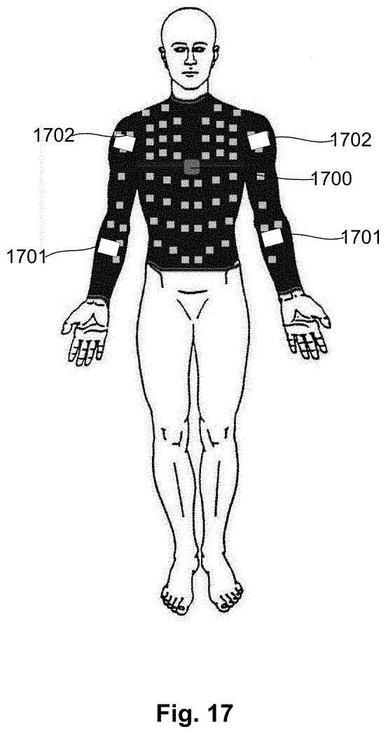

An example of the relative positions of the central processing units and tertiary processing units will be described with reference to FIG. 17.

FIG. 17 shows an example of a system 100 including a single location for the central processing units at 1700, two locations on the subject's forearms for some of the tertiary processing units at 1701 and two locations on the subject's upperarms for some of the tertiary processing units at 1702. At each location, there may be one or more processing units. For example, there may be four central processing units at 1700, two tertiary processing units at each 1701 and one tertiary processing unit at each 1702.

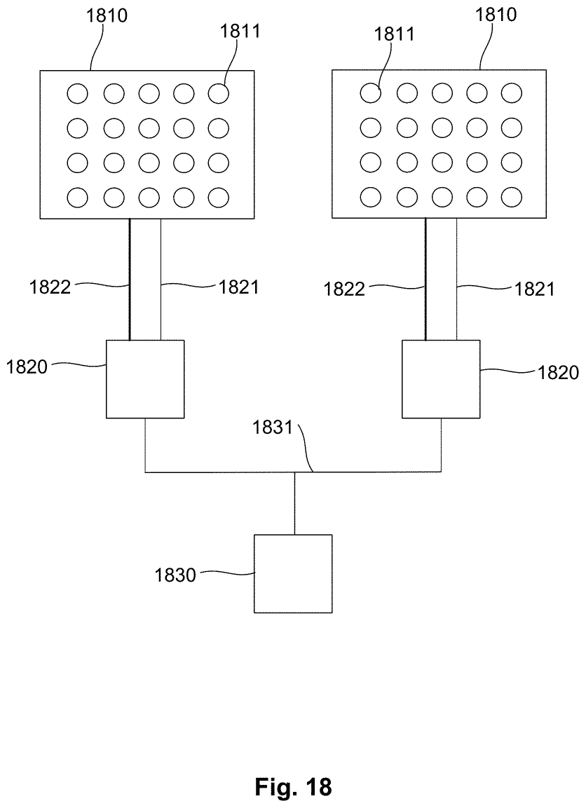

An example of the connection between the central processing units, tertiary processing units and arrays of electrodes will be described with reference to FIG. 18.

FIG. 18 shows an example of a system 100, including two electrode arrays 1810, each including a plurality of electrodes 1811, two tertiary processing units 1820 and the central processing unit 1830. Each electrode array 1810 is electrically connected to one of the tertiary processing units 1820 with a cable capable of transferring power 1821 and/or a cable capable of transferring data 1822. While connections 1821 and 1822 are demonstrated as two separate connections, a person skilled in the art would appreciate that they could be combined into a single cable. The tertiary processing units 1820 are then connected to the central processing units 1830 through cable 1831, allowing data to be transferred between them. While connections 1821, 1822 and 1831 are demonstrated as physical cables, a person skilled in the art would appreciate that the same effect could be achieved through well-known wireless methods.

Accordingly, in one example, the above described system determines how effectively and/or efficiently a subject is moving by classifying their activity and then comparing their performance to a healthy and/or idealised template. The system includes pose sensor(s), which allow for computer vision movement technique analysis, garment(s), which monitor the muscle activity of the subject, and processing devices that are configured to perform analysis and operate the system.

The system may have inbuilt knowledge of all bones, ligaments, muscles in the body and the angles at which they pull. Additionally, the system may know the healthy timing intervals for the activations of muscles during physical activities. In one example, this information is embodied as template, representing idealised muscle activation patterns and/or poses, allowing these to be easily compared to measured values to assess a subject's muscle activation and/or pose as they perform an activity.

The system may be able to identify a subject in frame and/or determine the outline and/or silhouette of the subject.

In one example, the system may be able to locate joints and determine the angle of joints of the upper limb, lower limb, cervical, thoracic and lumbar spine, pelvis, or the like. The system may do so using computer vision techniques, such as DeepPose, DensePose, VideoPose 3D, OpenPose algorithms and/or the like.

The system can also be able to determine the activation contributions of different muscles required to achieve the positions and torques of joints. Additionally, the system may be able to determine the stress/strain on tissue using the positions and torques of joints.

The system may be able to classify the physical activity being performed. The system may be able to determine how long the subject is performing the physical activity in an ideal and/or healthy technique.

The system may compare the parameters determined from the subject to the ideal and/or healthy template. The system may determine from this comparison how the subject's movements differ from the ideal and/or healthy template. For example, the system may determine that the subject is not performing a physical activity ideally if the subject is placing excessive strain on their muscles.

The system may outline torques being generated at joints, calculate muscle activation patterns, contribution weightings that enable joint movements, tissue stress, tissue strain and/or the like if the system is unable to classify the physical activity to a template. The system may compare these parameters to existing knowledge of tissue stresses and/or tissue strains to predict the risk of injury.

The system may output conclusions about the subject's technique, performance, issues and/or the like that are a problem. The system may suggest reasons as to why a subject is experiencing pathology and/or methods for minimising and/or eliminating the pathology. The system may also predict future pathology for the subject if they continue with their current and/or predicted technique.

In one example, the system analyses how a user is moving and may be configured to make the user aware of any current problems with their technique and/or problems that could potentially arise in the future. Muscle monitoring and data determined from the garment(s) can add accuracy to the system. By analysing data determined from the pose sensor(s) and garment(s), the subject can have access to a more accurate assessment of their muscle activations, strains and/or stresses on their muscles, their technique and/or performance. This can allow the system to make more accurate recommendations for technique changes to reduce stress and/or strain on tissue and the rate of degeneration.

The system may be configured to monitor at least one athlete during a sporting match. The system may include facial and/or player recognition that allow the system to be configured to monitor multiple individuals at the same time.

The system may include a facial and/or player recognition element that allows the system to track either a single player and/or a group of players where the imaging device may observe multiple individuals simultaneously. Additionally, the system may also perform subject localisation. For example, when tracking a group of players in a football match, the system may track the movements of the subjects which can be used to analyse the tactics of the subjects.

The system may monitor a subject while running. The subject may be running in a controlled environment (such as on a treadmill in a clinic) or in a chaotic environment (such as in a park where there are other individuals). Running requires many different muscles in specific coordination in order to minimise the stress/strain on tissue. Each muscle generates its own force and line of pull due to its specific attachment site which allows for joint movement. The amplitude, timing and frequency of the combined activation of these muscles contributes to the overall movement. If specific muscles are activating at the wrong time or to a lesser amplitude to which would be considered optimal during running, it can place excessive stress/strain on tissues which can lead to injury. If the subject's gluteal and other femoral external rotators and adductors do not activate to a sufficient amount during stance phase of running then the femur could internally rotate and increase the risk of rupturing the ACL, MCL as well as tear the meniscus.

The pose sensor(s) can monitor the subject's joint angles and the garment(s) monitor the subject's muscle activation amplitude and frequency, the data determined from the system can indicate when the subject may be at a higher risk of injury. For example, during a football match a coach could use this data to know when to substitute and rest the subject. In some cases, the pose sensor(s) may be able to identify a higher risk of injury without analysis from the garment(s). The accuracy and/or efficiency of the data determined by the pose sensor(s) may be improved by data provided by the garment(s).

Data determined by the pose sensor(s) and garment(s) may assist in diagnosing a subject's pain during running. If a subject uses the garment(s) during an extended run and they begin to feel pain in their calf part way through the run, then the garment(s) may determine that the gluteal muscles are reducing in their amplitude and frequency of activation with each stride. This would then cause the subject's brain to work out which other lower limb muscle could provide propulsion to move the subject forward and may use the gastrocnemius or soleus to achieve this goal. Since the calf is not used to contributing as much to the overall movement of the subject, it begins to fatigue and tighten causing excessive pulling in the achilles tendon and causing pain. Unless a clinician is experienced with this problem, they are unlikely to identify the issue without assistance.

The system may use a variety of algorithms to identify the subject pose, determine muscle activation patterns and analyse these parameters. The system may use convolutional neural networks that identify the locations of joints and link neighbouring joints together to determine whether the object is the subject. The system may also use temporal convolutional neural networks to identify the activity the subject is performing. The system may also use pattern recognition techniques and/or measures of similarity (such as cosine similarity) to compare joint positions to a healthy and/or idealised template. Each of these methods, and other suitable algorithms, are described in the prior art and are not described in further detail.

The system could be used (potentially in the form of a smart device with a camera) to monitor the subject as they run. The pose sensor(s) monitors the subject running and observes their technique over time. The pose sensor(s) may determine that the subject begins to show signs of reduced stability through femoral internal rotation or a mild Trendelenburg sign indicating the gluteal muscles are underactivating. The system may determine this before the excessive strain through the achilles tendon were to occur therefore preventing the subject from running so far as to commence pain, but also allowing awareness that the pain would have occurred if they were to continue. In addition, the garment(s) may provide the subject with information about the gluteal region underactivating and the calf overactivating. The system 100 may assist in identifying which muscles must be underactivating due to their unique line of pull.

The pose sensor(s) and garment(s) may be used independently, allowing the subject to use the system where it may otherwise be inconvenient, impractical and/or impossible to do so otherwise. For example, a subject may go for a run where they are unable to use an imaging device to monitor their pose and/or technique. In this case, the subject is still able to use the garment(s) to perform analysis on their muscle activation patterns. Conversely, a subject may go rock climbing where the garment(s) may interfere with the safety harness required to perform the activity safely. In this case, the subject may still perform their activity in view of the pose sensor(s) so it may determine the subject's pose.

The system may be used to assist in developing a treatment strategy for subjects with neurological conditions such as cerebral palsy. Individuals with cerebral palsy may move with a crouched gait pattern showing excessive femoral internal rotation at the femur as well as inability to extend the knees due to shortened hamstrings. When watching a subject with cerebral palsy walk, it can be difficult during the limited time of assessment for clinicians to immediately identify which muscles are over/under activating and thus the most optimal method of treatment for the subject. Occasionally, muscle lengthening surgeries are available to allow a subject to walk with a less crouched, more upright pattern. The pose sensor(s), could independently or in conjunction with the garment(s), assist in identifying the most optimal method of mobilisation for the subject which reduces the rate of stress/strain on tissues as well as allows for the slowest rate of degeneration for joints by calculating an optimal muscle contribution ratio.

The system may be used to perform analysis of the subject while performing a wide variety of exercises. Firstly, the subject may log in to the system, allowing the system to access information pertinent to the subject. This information may include the past and current physical conditions of the user and/or future physical and/or fitness goals of the subject. Alternatively, the system may identify the subject based on their physical attributes and automatically retrieve information pertinent to the subject. Before the subject begins their physical activity, the subject or an instructor may select the physical activity they wish to perform. Alternatively, the subject may not select the physical activity and allow the system to automatically identify what physical activity the subject performs.

While the subject performs their physical activity, the system monitors the subject using a camera and may also receive information from sensors located on the subject's body which determines muscle indicators. As the system monitors the subject, it performs an analysis, identifying the location of the subject's joints, how the subject's activity should be classified, the number of repetitions the subject performs and compares this analysis to a healthy or idealised template performing the same or similar physical activity.

As the subject is performing the physical activity, the system may provide feedback to the subject in real time or with a short delay. This feedback could include information on the subject's technique and how to improve it, or to suggest that the subject changes their physical activity, such as increasing or reducing the speed the physical activity is performed with or suggesting the subject performs a different physical activity. The subject can also provide feedback to the system while performing an activity, for example, the subject may tap a region of their body that feels pain during a physical activity. This can direct the system to perform a more in-depth analysis of the subject's technique near the area where pain was felt, indicate to the subject how ideal their technique was and potentially suggest how the technique can be modified to avoid pain in the future.

The subject could tap either on their own body, the location where they are feeling pain, or on a touchscreen device, such as a smartphone, tablet etc., to indicate where they are feeling pain. Depending on the implementation of the system, the subject may be required to tap multiple times or a single time for the system to detect that the subject is indicating there is a painful location on their body. After the system detects the subject's tapping, the system will perform an analysis to determine what may be causing the subject pain. Initially, the system may look to measured stress/strain amounts of the subject but may use other factors if the results from the stress/strain analysis are inconclusive.

The system may also receive feedback from the subject after the physical activity has been completed as it may be impractical for the subject to indicate during physical activity that a section of their body feels pain. The system can then show the subject a recording of their physical activity and allow the subject to indicate to the system what part of their body felt pain while watching a replay of their technique.

The subject may also supply written or oral notes to the system, which the system can attempt to decipher these notes and provide further feedback to the subject. If the system is not confident in the consistency and/or accuracy of the feedback it may supply to the subject, which may be caused by incorrect placement of the camera or defects in the camera, the system may indicate to the subject that is has low confidence in the results and indicate to the subject how it may assist the system to correct any issues.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

Persons skilled in the art will appreciate that numerous variations and modifications will become apparent. All such variations and modifications which become apparent to persons skilled in the art, should be considered to fall within the spirit and scope that the invention broadly appearing before described.

The claims defining the invention are as follows:

1. A system for monitoring a physical activity performed by a subject, the system including:
a. a pose sensor configured to sense one or more pose parameters at least partially indicative of a subject pose;
b. a garment including a number of arrays of electrodes positioned on the garment so that when the garment is worn by the subject, the electrodes contact skin of the subject and generate electrical signals indicative of electrical potentials within respective muscles of the subject, each array of electrodes including a plurality of electrodes arranged in a grid; and,
c. one or more processing devices configured to:
i. use signals from the electrodes to determine one or more muscle activation patterns;
ii. use the pose parameters to at least partially determine the subject pose; and,
iii. determine an activity indicator indicative of the physical activity at least partially based on the determined one or more muscle activation patterns and at least partially based on the determined subject pose.

2. A system according to claim 1, wherein the pose parameters are indicative of at least one of:
a. a location of one or more limbs of the subject;
b. a location of one or more joints of the subject; and,
c. an angle of one or more joints of the subject.

3. A system according to any one of claim 1 or 2, wherein the pose sensor includes an imaging device configured to capture one or more images of the subject when performing the physical activity and wherein the one or more processing devices are configured to:
a. receive image data indicative of the one or more images from the pose sensor; and,
b. analyse the image data to determine the pose parameters.

4. A system according to any one of the preceding claims, wherein the one or more processing devices analyse the image data at least in part by identifying at least one of:
a. an outline of the subject;
b. a silhouette of the subject;
c. segments of the subject; and,
d. the subject.

5. A system according to any one of the preceding claims, wherein the garment includes at least one marker and wherein the pose sensor is configured to detect the at least one marker.

6. A system according to any one of the preceding claims, wherein the one or more processing devices include:
a. one or more central processing units; and,
b. one or more tertiary processing units.

7. A system according to claim 6, wherein the one or more tertiary processing units are configured to at least partially process at least some of the signals from the electrodes.

8. A system according to claim 6 or claim 7, wherein the one or more tertiary processing units are configured to transmit signal data at least partially indicative of the signals from the electrodes to the one or more central processing units.

9. A system according to claim 7 or claim 8, wherein the one or more central processing units are configured to receive and process the signal data.

10. A system according to any one of claims 7 to 9, wherein the garment includes each of the one or more tertiary processing units on or near a different muscle group of the subject.

11. A system according to any one of claims 7 to 10, wherein each array of electrodes includes a respective tertiary processing unit.

12. A system according to any one of claims 7 to 11, wherein each of the one or more tertiary processing units at least partially processes signals from electrodes associated with different muscle groups of the subject.

13. A system according to any one of the preceding claims, wherein the pose sensor includes at least one of:
a. a position sensor configured to measure a position of at least part of the subject;
b. a movement sensor configured to measure movement of at least part of the subject;
c. a strain sensor incorporated into the garment and configured to measure strain of the garment; and,
d. a pressure sensor configured to measure pressure imparted by at least part of the subject.

14. A system according to any one of the preceding claims, wherein the one or more processing devices are configured to determine the muscle activations that contribute to at least one of:
a. a joint position;
b. a joint torque;
c. a joint angle;
d. a tissue stress; and,
e. a tissue strain.

15. A system according to any one of the preceding claims, wherein the system includes one or more pose templates indicative of reference poses and wherein the one or more processing devices are configured to:
a. retrieve a pose template;
b. compare the pose parameters to the pose template; and,
c. using results of the comparison to determine at least one of:
i. a pose indicator indicative of the subject pose;
ii. the activity indicator; and,
iii. the physical activity.

16. A system according to claim 15, wherein the pose templates are indicative of idealized poses associated with the physical activity.

17. A system according to any one of the preceding claims, wherein the system includes one or more muscle templates indicative of reference muscle activation patterns and wherein the one or more processing devices are configured to:
a. retrieve one or more muscle templates;
b. compare the signals from the electrodes to the one or more muscle templates; and,
c. using results of the comparison to determine at least one of:
i. a muscle activity indicator indicative of muscle activation;
ii. the activity indicator; and,
iii. the physical activity.

18. A system according to claim 17, wherein the muscle templates are indicative of idealized muscle activation patterns associated with the physical activity.

19. A system according to any one of preceding claims, wherein the system includes one or more activity templates indicative of reference physical activity and wherein the one or more processing devices are configured to:
a. retrieve an activity template;
b. compare the subject pose and muscle activation patterns to the activity template; and,
c. using results of the comparison to determine at least one of:
i. the activity indicator; and,
ii. the physical activity.

20. A system according to claim 19, wherein the activity templates are indicative of idealized muscle activity patterns and idealized poses associated with the physical activity.

21. A system according to any one of claims 15 to 20, wherein the templates are determined based on at least one of:
a. measurements previously recorded for the subject; and,
b. measurements recorded for one or more reference subjects.

22. A system according to any one of claims 15 to 21, wherein the one or more processing devices are configured to retrieve a template at least partially based on at least one of:
a. pose parameters;
b. muscle activation patterns;
c. an indication of the physical activity;
d. user input commands; and,
e. one or more physical characteristics of the subject.

23. A system according to claim 22, wherein the subject's physical characteristics are at least one of the subject's:
a. height;
b. weight; and,
c. body proportions.

24. A system according to claim 22 or 23, wherein the one or more processing devices determine the physical characteristics based on user input commands.

25. A system according to any one of the preceding claims, wherein the one or more processing devices are configured to determine the physical activity using at least one of:
a. user input commands;
b. pose parameters; and,
c. muscle activation patterns.

26. A system according to any one of claims 15 to 25, wherein the templates include:
a. short activity duration templates;
b. extended activity duration templates;
c. high activity intensity templates; and,
d. low activity intensity templates.

27. A system according to any one of claims 15 to 26, wherein the one or more processing devices are configured to:
a. determine one or more physiological parameters;
b. compare the one or more physiological parameters to the template; and,
c. determine the activity indicator using the results of the comparison.

28. A system according to claim 27, wherein the physiological parameters include measuring at least one of:
a. lactic acid;
b. impedance;
c. hydration levels;
d. sweat composition;
e. ion composition;
f. blood glucose; and,
g. heart rate.

29. A system according to any one of claims 15 to 28, wherein the one or more processing devices are configured to use machine learning to at least one of:
a. identify the physical activity;
b. classify the physical activity;
c. select a template;
d. generate a template; and,
e. perform a comparison.

30. A system according to any one of claims 15 to 29, wherein the comparison includes data on at least one of the subject's:
a. joint coordinates;
b. muscle activation patterns;
c. performance;
d. technique; and,
e. pathology.

31. A system according to any one of claims 15 to 30, wherein the comparison is based on at least one of:
a. a subject fatigue;
b. a degradation in subject performance; and,
c. a change in risk of subject pathology.

32. A system according to any one of the preceding claims, wherein the one or more processing devices are configured to predict future subject pathology.

33. A system according to any one of the preceding claims, wherein the one or more processing devices are configured to derive one or more muscle activation parameters.

34. A system according to claim 33, wherein the one or more processing devices are configured to compare the muscle activation parameters to reference muscle activation parameters.

35. A system according to claim 34, wherein the one or more muscle activation parameters and reference muscle activation parameters are at least one of:
a. tissue stresses; and,
b. tissue strains.

36. A system according to claim 34 or 35, wherein the reference muscle activation parameters are at least one of:
a. based on commonly known muscle parameters;
b. based on subject physical characteristics;
c. determined using machine learning; and,
d. determined using user input commands.

37. A method for monitoring a physical activity performed by a subject, the method including:
a. using a pose sensor to sense one or more pose parameters at least partially indicative of a subject pose;
b. using a garment including a number of arrays of electrodes positioned on the garment so that when the garment is worn by the subject, the electrodes contact skin of the subject and generate electrical signals indicative of electrical potentials within respective muscles of the subject, each array of electrodes including a plurality of electrodes arranged in a grid; and,
c. in one or more processing devices, determining:
i. one or more muscle activation patterns at least partially using signals from the electrodes;
ii. the subject pose at least partially using the pose parameters; and,
iii. an activity indicator indicative of the physical activity at least partially based on the determined one or more muscle activation patterns and at least partially based on the determined subject pose.

38. A system for monitoring a physical activity performed by a subject, the system including:
a. a pose sensor configured to sense one or more pose parameters at least partially indicative of a subject pose; and,
b. one or more processing devices configured to:
i. use the pose parameters to at least partially determine the subject pose; and,
ii. determine an activity indicator indicative of the physical activity at least partially based on the determined subject pose.

39. A system according to claim 38, wherein the pose parameters are indicative of at least one of:
a. a location of one or more limbs of the subject;
b. a location of one or more joints of the subject; and,
c. an angle of one or more joints of the subject.

40. A system according to any one of claim 38 or 39, wherein the pose sensor includes an imaging device configured to capture one or more images of the subject when performing the physical activity and wherein the one or more processing devices are configured to:
a. receive image data indicative of the one or more images from the pose sensor; and,
b. analyse the image data to determine the pose parameters.

41. A system according to any one of claims 38 to 40, wherein the one or more processing devices analyse the image data at least in part by identifying at least one of:
a. an outline of the subject;
b. a silhouette of the subject;
c. segments of the subject; and,
d. the subject.

42. A system according to any one of claims 38 to 41, wherein the pose sensor includes at least one of:
a. a position sensor configured to measure a position of at least part of the subject;
b. a movement sensor configured to measure movement of at least part of the subject;
c. a strain sensor incorporated into the garment and configured to measure strain of the garment; and,
d. a pressure sensor configured to measure pressure imparted by at least part of the subject.

43. A system according to any one of claims 38 to 42, wherein the system includes one or more pose templates indicative of reference poses and wherein the one or more processing devices are configured to:
a. retrieve a pose template;
b. compare the pose parameters to the pose template; and,
c. using results of the comparison to determine at least one of:
i. a pose indicator indicative of the subject pose;
ii. the activity indicator; and,
iii. the physical activity.

44. A system according to claim 43, wherein the pose templates are indicative of idealized poses associated with the physical activity.

45. A method for monitoring a physical activity performed by a subject, the method including:
a. using a pose sensor to sense one or more pose parameters at least partially indicative of a subject pose;
b. in one or more processing devices, determining:
i. one or more muscle activation patterns at least partially using signals from the electrodes;
ii. the subject pose at least partially using the pose parameters; and,
iii. an activity indicator indicative of the physical activity at least partially based on the determined one or more muscle activation patterns and at least partially based on the determined subject pose.
